(19) United States
(12) Patent Application Publication
(10) Pub. No.: US 2024/0058234 A1
(43) Pub. Date: Feb. 22, 2024

(54) COMPOSITIONS AND RELATED METHODS

(71) Applicant: Colgate-Palmolive Company, New York, NY (US)

(72) Inventors: Xiang CHEN, Somerset, NJ (US); Brandon ZOELLNER, Whitehouse Station, NJ (US); Suman CHOPRA, Monroe, NJ (US); Venda Porter MALONEY, Metuchen, NJ (US); Shuyun WU, Guangzhou (CN)

(21) Appl. No.: 18/354,302

(22) Filed: Jul. 18, 2023

(30) Foreign Application Priority Data
Aug. 17, 2022 (CN) 202210985240.6

(51) Int. Cl. A61K 8/23; A61K 8/02; A61Q 11/00

(52) U.S. Cl. CPC A61K 8/23; A61K 8/0237; A61Q 11/00; A61K 2800/87; A61K 2800/56; A61K 2800/92

(57) ABSTRACT

The present disclosure provides an intermediate composition (e.g., an aqueous slurry) for the formulation of a tooth whitening strip comprising a hydratable adhesive film, wherein the intermediate composition comprises an inorganic salt of peroxymonosulfate, preferably an alkali metal salt or alkaline earth metal salt, or mixtures thereof, and methods of using the same and making the tooth whitening strip.

### CROSS-REFERENCE TO RELATED APPLICATIONS

This application is an U.S. nonprovisional application which claims priority to, and the benefit of, Chinese Application No. 202210985240.6, filed on Aug. 17, 2022, the contents of which is hereby incorporated by reference in its entirety.

### BACKGROUND

Consumer products providing for teeth whitening are numerous and take many forms, but one of the more popular forms are dental whitening strips. These strips, commonly with an adhesive material, are typically adhered to the anterior surface of the teeth for a prolonged period of time, such as 10 minutes or more, during which time an entrained whitening agent diffuses into the tooth enamel to bleach away stains. Such strips are often more convenient and more effective than alternative at-home whitening treatments, such as toothpastes and mouthwashes, because of the prolonged contact provided between the teeth and the whitening composition.

Products that are presently available to whiten teeth include a variety of different ingredients, and the primary active ingredient is most commonly a peroxide source such as hydrogen peroxide. The use of peroxide agents often presents numerous difficulties in both formulation and long-term stability of the resulting compositions. In addition, in high concentrations, or in prolonged contact with the oral mucosa, hydrogen peroxide can be highly irritating to the teeth and gums. Thus, alternative oxidizing agents with improved stability are needed, especially for whitening products which provide long-term contact with oral tissues.

Peroxysulfuric acid, and its salts, the peroxysulfates, are powerful oxidizing and stain removing agents. They are currently used for a variety of industrial and consumer purposes, including swimming pool treatment and denture cleaning. Peroxysulfate whitening products have been explored for some oral care applications, such as mouthwashes and toothpastes. The most common peroxymonosulfate oxidizing agent is potassium peroxymonosulfate, commonly referred to as MPS, and sold as part of the composition Oxone or Caroat.

The use of potassium monoperoxysulfate in oral care applications has been very limited by its instability in aqueous solution, especially in aqueous solution near or above neutral pH. Potassium monoperoxysulfate has been known to degrade even in the presence of small quantities of water and heat. Thus, potassium monoperoxysulfate whitening compositions face particular difficulties in formulations intended for anything more than transient or momentary use in the oral cavity.

There is thus a need for tooth whitening products which provide the convenience and duration of a strip with improved properties of peroxysulfate whitening agents.

### BRIEF SUMMARY

In a first aspect, the present disclosure provides an intermediate composition (Composition 1) (e.g., an aqueous slurry) for the formulation of a tooth whitening strip comprising a hydratable adhesive film, wherein the intermediate composition comprises an inorganic salt of peroxymonosulfate, preferably an alkali metal salt or alkaline earth metal salt, or mixtures thereof.

In a second aspect, the present disclosure provides a tooth whitening strip (Strip 1), for example, comprising or made using the intermediate composition (Composition 1).

Further areas of applicability of the present disclosure will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

Open terms such as “include,” “including,” “contain,” “containing” and the like mean “comprising.” In this description, unless otherwise stated, the use of the singular also includes the plural. For example, “a lubricant” also comprehends the case where more than one lubricant is used

In a first aspect, the present disclosure provides an intermediate composition (Composition 1) (e.g., an aqueous slurry) for the formulation of a tooth whitening strip comprising a hydratable adhesive film, wherein the intermediate composition comprises an inorganic salt of peroxymonosulfate, preferably an alkali metal salt or alkaline earth metal salt, or mixtures thereof. In further embodiments, the present disclosure provides (all percentages by weight provided hereinbelow are measured by weight of the intermediate composition):
1.1. Composition 1, wherein the inorganic salt of peroxymonosulfate is sodium peroxymonosulfate, potassium peroxymonosulfate, or lithium peroxymonosulfate, or a combination thereof;
1.2. Composition 1 or 1.1, wherein the salt of peroxymonosulfate is potassium peroxymonosulfate;
1.3. Composition 1.2, wherein the potassium peroxymonosulfate is provided as a triple salt of potassium peroxymonosulfate, potassium hydrogen sulfate and potassium sulfate (e.g. Caroat®), optionally wherein the triple salt comprises about 45%-50% by weight of potassium peroxymonosulfate (MPS), e.g., 47% or 49% by weight of potassium peroxymonosulfate;
1.4. Any of the preceding Compositions, wherein the composition comprises an effective amount of peroxymonosulfate salt which is 0.01% to 5%, by weight of the composition, e.g., 0.05% to 5%, or 0.1% to 5%, or 0.5% to 3%, or 0.5% to 2.5%, or 0.5% to 2%, or 0.5% to 1.5%, or 0.75% to 1.25%, or 0.9% to 1.2%, or 0.6% to 1.2%, or 0.4% to 2%, or 0.4% to 1.5%, or 0.4% to 1.2%, or 0.4% to 1%, or 0.4% to 0.8%, or 0.3% to 2.0%, or 0.3% to 1.5%, or 0.3% to 1.2%, or 0.3% to 0.9%, or about 1%, or about 0.8%, or about 0.6%, by weight of the composition;
1.5. Any of the preceding compositions, wherein the composition further comprises a second inorganic salt of peroxymonosulfate;
1.6. Any of the preceding compositions, wherein the inorganic peroxymonosulfate salt is not particulated (e.g., not granulated);
1.7. Any of the preceding compositions, wherein the composition does not comprise hydrogen peroxide;
1.8. Any of the preceding compositions, wherein the composition does not comprise any of hydrogen peroxide, urea peroxide, peroxide salts (e.g., sodium peroxide, potassium peroxide, lithium peroxide, calcium peroxide), peroxy acids (e.g., peroxyacetic acid, peroxybenzoic acid, or salts or derivatives thereof), organic peroxides (e.g., urea hydrogen peroxide, glyceryl hydrogen peroxide, peroxy esters, diacyl peroxides, monoperoxyphthalate, or salt thereof), perborate salts, persilicate salts, percarbonate salts, chlorinated oxidizing agents (e.g., hypochlorite salts, chlorite salts, chlorate salts, perchlorate salts, chlorine dioxide);
1.9. Any of the preceding compositions, wherein the inorganic salt of peroxymonosulfate is the only oxidizing agent present in the composition;
1.10. Composition 1.9, wherein potassium peroxymonosulfate (e.g., as part of a triple salt mixture) is the only oxidizing agent present in the composition;
1.11. Composition 1, or any of 1.1-1.10, wherein the composition comprises one or more water-soluble or water-swellable polymers, including anionic polymers and/or neutral polymers;
1.12. Composition 1.11, wherein the composition comprises from 5%-50% by weight of water-soluble or water-swellable polymers or humectants, for example, 5%-30% by weight, or 10-30% by weight, or 10-25% by weight, or 15-25% by weight, or 20-30% by weight, or 25-30% by weight, (e.g., about 20%, or about 25%, or about 30%, or about 27% by wt.);
1.13. Composition 1, or any of 1.1-1.12, wherein the composition comprises one or more humectants selected from: polyethylene glycols (such as PEG-200, PEG-300, PEG-400, PEG-500, PEG-600, PEG-800, PEG-1000, PEG-1600, PEG-2000), polypropylene glycols, polyoxyethylene-polyoxypropylene copolymers (PEG-PPG, including block copolymers, triblock copolymers (poloxamers, such as poloxamer 407), and random copolymers, such as PEG/PPG-116/66 and PEG/PPG-38/8), ethanol, glycerin, propylene glycol, sorbitol, and xylitol;
1.14. Composition 1.13, wherein the composition comprises one or more humectants selected from polyethylene glycols, PEG-PPG copolymers, glycerin, and propylene glycol;
1.15. Composition 1.14, wherein the composition comprises one or more humectants selected from polyethylene glycols, PEG-PPG copolymers, and glycerin;
1.16. Composition 1.14, wherein the composition comprises at least one polyethylene glycol, e.g., selected from PEG-200, PEG-300, PEG-400, PEG-500, PEG-600, PEG-800, PEG-1000, PEG-1600, and PEG-2000;
1.17. Composition 1.16, wherein the composition comprises PEG-600;
1.18. Any of Compositions 1.13-1.17, wherein the composition comprises at least one PEG-PPG copolymer;
1.19. Composition 1.18, wherein the PEG-PPG copolymer is a random copolymer, e.g., PEG/PPG-116/66 or PEG/PPG-38/8;
1.20. Composition 1.18, wherein the PEG-PPG copolymer is a triblock copolymer. i.e., a poloxamer;
1.21. Composition 1.20, wherein the poloxamer is selected from Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, and Poloxamer 407;
1.22. Composition 1.21, wherein the poloxamer is selected from Poloxamer 105 and Poloxamer 407;
1.23. Composition 1.22, wherein the poloxamer is Poloxamer 105;
1.24. Any of Compositions 1.13-1.23, wherein the composition comprises a total of not more than 10% by weight of said humectants, for example, not more than 8% by weight, not more than 7% by weight, nor more than 6% by weight, not more than 5% by weight, not more than 4% by weight, or not more than 3% by weight, or not more than 2.5% by weight, or not more than 2% by weight, or not more than 1% by weight, of said humectants;
1.25. Any of Composition 1.13-1.24, wherein the composition comprises from 0.5% by weight to 10% by weight of said humectants, for example, 0.5% to 8% by weight, or 0.5% to 7% by weight, or 0.5 to 6% by weight, or 0.5% to 5% by weight, 0.5% to 4% by weight, or 0.5% to 3% by weight, or 0.5% to 2.5% by weight, or 0.5% to 2% by weight, or 1% to 5% by weight, or 1% to 4% by weight, or 1% to 3% by weight, or 1% to 2.5% by weight, or 1% to 2% by weight, or 1.5% to 5% by weight, or 1.5% to 4% by weight, or 1.5% to 3% by weight, or 1.5% to 2.5% by weight, or about 2% by weight;
1.26. Composition 1.25, wherein the composition comprises 0.5% by weight to 10% by weight of polyethylene glycols (e.g. PEG-600), for example, 0.5% to 8% by weight, or 0.5% to 7% by weight, or 0.5 to 6% by weight, or 0.5% to 5% by weight, 0.5% to 4% by weight, or 0.5% to 3% by weight, or 0.5% to 2.5% by weight, or 0.5% to 2% by weight, or 1% to 10% by weight, or 1% to 8% by weight, or 1% to 6% by weight, or 1% to 5% by weight, or 1% to 4% by weight, or 1% to 3% by weight, or 1% to 2.5% by weight, or 1% to 2%, or 1.5% to 3%, or 1.5% to 2.5%, or 1.5% to 2%, or 3% to 8% by weight, or about 2%, of polyethylene glycols.
1.27. Composition 1.26, wherein the polyethylene glycols comprise PEG-200, PEG-300, PEG-400, PEG-500, PEG-600, PEG-800, or a combination thereof;
1.28. Composition 1.27, wherein the polyethylene glycols comprise PEG-600;
1.29. Any of Compositions 1.13-1.28, wherein the composition does not comprise ethanol;
1.30. Any of Compositions 1.13-1.29, wherein the composition does not comprise sorbitol or xylitol;
1.31. Any of Compositions 1.13-1.30, wherein the composition does not comprise glycerol, or propylene glycol;
1.32. Any of Compositions 1.13-1.31, wherein the composition does not comprise propylene glycol;
1.33. Any of Compositions 1.13-1.32, wherein the composition does not comprise PEG-PPG copolymers;
1.34. Any of Compositions 1.13-1.33, wherein a polyethylene glycol (e.g., PEG-600) is the only humectant;
1.35. Composition 1, or any of 1.1-1.34, wherein the composition further comprises one or more polymers selected from polysaccharides (e.g., cellulose derivatives, for example carboxymethyl cellulose, hydroxyethyl cellulose, hydroxypropyl cellulose, hydroxypropyl methyl cellulose, ethyl cellulose, microcrystalline cellulose; or polysaccharide gums, for example xanthan gum, guar gum, or carrageenan gum, pectins, karaya gum); chitosans; dextrans; polyvinyl pyrrolidone (PVP), such as cross-linked PVP; hyaluronic acid and sodium hyaluronates; synthetic anionic polymeric polycarboxylates, such as copolymers of maleic anhydride or acid with another polymerizable ethylenically unsaturated monomer, preferably methyl vinyl ether (e.g., copolymers in a 1:4 to 4:1 ratio of maleic anhydride/acid to methyl vinyl ether); polyphosphonic acids and polyphosphonates (i.e., polyphosphoesters); cross-linked carboxyvinyl copolymers; polyacrylic acid or polyacrylate polymers; polyacrylamides, such as (2-hydroxypropyl)methacrylamide; polyamines; polyvinyl alcohols; polyvinyl pyrrolidine-polyvinyl acetate copolymers (PVP-VA); polyoxazolines, such as poly(2-alkyl-2-oxazolines), e.g., methyl, ethyl, or isopropyl substituted polyoxazolines; and quaternary ammonium polymers;
1.36. Composition 1, or any of 1.1-1.35, wherein the composition comprises one or more of: polyethylene glycol polymers (PEG), polyacrylic acid or polyacrylate polymers (PAA), polyvinylpyrrolidone polymers (PVP), polyvinylpyrrolidone-vinyl acetate copolymers (PVP-VA), polyoxazoline polymers (PO), polyoxyethylene-polyoxypropylene (PEG-PPG) block copolymers, and mixtures thereof;
1.37. Composition 1.36, wherein the composition comprises at least one of each of: a polyethylene glycol polymer, a polyacrylic acid or polyacrylate polymer, a polyvinylpyrrolidone polymer, a polyvinylpyrrolidone-vinyl acetate copolymer, a polyoxazoline polymer, and a polyoxyethylene-polyoxypropylene copolymer;
1.38. Composition 1, or any of 1.1-1.37, wherein the composition comprises at least one of: a cellulose derivative (e.g., carboxymethyl cellulose, hydroxyethyl cellulose, hydroxypropyl cellulose, hydroxypropyl methyl cellulose, or ethyl cellulose), a polyethylene glycol polymer (PEG), a polyacrylic acid or polyacrylate polymer (PAA), a polyvinylpyrrolidone polymer (PVP), a polyvinylpyrrolidone-vinyl acetate copolymer (PVP-VA), a polyoxazoline polymer (PO), a polyoxyethylene-polyoxypropylene (PEG-PPG) block copolymer, or mixtures thereof;
1.39. Composition 1, or any of 1.28-1.38, wherein the composition comprises less than 30% by weight, of any of said polymers or mixtures of polymers, e.g., 1% to 30%, or 1% to 20%, or 1% to 15%, or 1% to 10%, or 1% to 7%, or 5% to 30%, or 5% to 25%, or 5% to 20%, or 5% to 15%, or 10% to 30%, or 10% to 25%, or 10% to 20%, or 15% to 30%, or 15% to 25%, or 15% to 20%, or about 25%, or about 20%, or about 15%, or about 12.5%, or about 10%, or about 6%, or about 5%;
1.40. Composition 1, or any of 1.1-1.39, wherein the composition comprises one or more polyvinylpyrrolidone (PVP) polymers, e.g., linear and/or cross-linked PVP polymers;
1.41. Composition 1.40, wherein the composition comprises linear PVP polymers, e.g., one or two linear PVP polymers;
1.42. Composition 1.41, wherein the linear PVP polymers are selected from PVP K90 and PVP K29/32, or a combination thereof;
1.43. Any of Compositions 1.40-1.42, wherein the composition comprises 10% to 40% by weight of PVP polymers, e.g., 10% to 35% by weight, or 10% to 30% by weight, or 10% to 25% by weight, or 15% to 40% by weight, or 15% to 35% by weight, or 15% to 30% by weight, or 15% to 25% by weight, or 20% to 40% by weight, or 20% to 35% by weight, or 20% to 30% by weight, or about 25% by weight;
1.44. Composition 1, or any of 1.1-1.43, wherein the composition does not comprise a polysaccharide polymer or derivative thereof;
1.45. Composition 1, or any of 1.1-1.43, wherein the composition does not comprise carboxymethyl cellulose, hydroxyethyl cellulose, hydroxypropyl cellulose, hydroxypropyl methyl cellulose, ethyl cellulose;
1.46. Composition 1, or any of 1.1-1.43, wherein the composition does not comprise hydroxypropyl methyl cellulose;
1.47. Composition 1, or any of 1.1-1.43, wherein the composition does not comprise carboxymethyl cellulose;
1.48. Composition 1, or any of 1.1-1.47, wherein the composition further comprises a polyphosphate or an organic cyclic polyphosphate, such as an alkali metal pyrophosphate, an alkali metal tripolyphosphate, an alkali metal tetraphosphate, an alkali metal hexametaphosphate, an alkali metal insoluble metaphosphate, an alkali metal phytic acid salt, or a mixture thereof;
1.49. Composition 1.48, wherein the composition comprises sodium or potassium pyrophosphate (e.g., tetrasodium or tetrapotassium pyrophosphate and/or di sodium or dipotassium pyrophosphate and/or sodium acid pyrophosphate), sodium or potassium tripolyphosphate, sodium or potassium tetraphosphate, sodium or potassium phytic acid salt, or a mixture thereof;
1.50. Composition 1.48 or 1.49, wherein the composition comprises from 0.05 to 5% by weight of polyphosphates, e.g., 0.05 to 2%, or 0.05% to 1%, or 0.05% to 0.5% or about 0.2% by weight of polyphosphates;
1.51. Composition 1, or any of 1.1-1.50, wherein the composition further comprises a desensitizing agent, e.g., in an amount from 0.1 to 5% by weight, such as potassium nitrate;
1.52. Composition 1, or any of 1.1-1.51, wherein the composition further comprises an enamel strengthening agent, e.g., in an amount from 0.1 to 5% by weight, such as zinc phosphate;
1.53. Composition 1 or any of 1.1-1.52, wherein the composition further comprises one or more of flavors and sweeteners;
1.54. Composition 1 or any of 1.1-1.53, wherein the composition comprises water, e.g., from 25%-85% by wt. of the composition (e.g., aqueous slurry);
1.55. Composition 1, or any of 1.1-1.54, wherein the composition consists of water, PVP, potassium peroxymonosulfate triple salt (e.g., Caroat®), and one or more of polyethylene glycol (e.g., PEG-600), PEG-PPG copolymer (e.g., Poloxamer 105), glycerin;
1.56. Composition 1, or any of 1.1-1.54, wherein the composition consists of water, PVP, potassium peroxymonosulfate triple salt (e.g., Caroat®), and polyethylene glycol (e.g., PEG-600);
1.57. Composition 1 or any of 1.1-1.56, wherein the composition is an aqueous slurry that comprises or consists of:

| | Amount by |
|---|---|
| Component | wt. % |
| MPS (e.g., provided as | 0.5-2% |
| Caroat ®, 45% MPS) | (e.g., 1%) |
| Water | q.s. (e.g., |
| | about 75%) |
| PVP | 10-20% |
| PEG 600 | 3-8% |
| Chelating agent | 0.1-1% |
| pH adjusting agent | 0.5-3% |
| Alkali metal pyrophosphate | 0.1-1% |

1.58. Composition 1 or any of 1.1-1.56, wherein the composition is an aqueous slurry that comprises or consists of:

| | Amount by |
|---|---|
| Component | wt. % |
| MPS (e.g., provided as | 0.3-1.5% |
| Caroat ®, 45% MPS) | (e.g., 0.6%) |
| Water | q.s. (e.g., |
| | about 60- |
| | 80%) |
| PVP (e.g., PVP K90 and/or | 15-30% |
| PVP K29/32) | (e.g., 25%) |
| PEG (e.g., PEG 600) | 0.5-5% (e.g., |
| | 2%) |
| pH adjusting agent (e.g. | 0.5-3% |
| NaOH) | |

or

| | Amount by |
|---|---|
| Component | wt. % |
| MPS (e.g., provided as | 0.5-3% (e.g., |
| Caroat ®, 45% MPS) | 0.9%) |
| Water | q.s. (e.g., |
| | about 60- |
| | 80%) |
| PVP (e.g., PVP K90 and/or | 15-30% |
| PVP K29/32) | (e.g., 25%) |
| PEG (e.g., PEG 600) | 0.5-5% (e.g., |
| | 2%) |
| pH adjusting agent (e.g. | 0.5-3% |
| NaOH) | |

1.59. Composition 1, or any of 1.1-1.58, wherein the composition is prepared as an aqueous suspension or aqueous/alcoholic suspension and subsequently dried onto a backing layer to form a tooth whitening strip;
1.60. Composition 1, or any of 1.1-1.58, wherein the composition is prepared as an aqueous suspension or aqueous/alcoholic suspension and subsequently dried onto a backing layer to form a tooth whitening strip, and wherein the tooth-whitening strip further comprises an inert removable paper or plastic protective film and/or an affixed inert paper or plastic backing composition and wherein said protective film or backing composition is adhered to the hydratable adhesive film, optionally, wherein the protective film and/or the backing composition are comprises of wax paper, coated paper, treated paper, polyethylene, polystyrene, polypropylene, polyvinyl chloride, polyvinyl acetate, polymethyl methacrylate, polytetrafluoroethylene, polyesters (e.g., polyethylene terephthalate), polyimides, polycarbonates, or any combination thereof;
1.61. Composition 1.60, wherein the protective film and/or the backing layer of the tooth-whitening strip comprises polypropylene, polyethylene, polyethylene terephthalate, and/or polystyrene;
1.62. Composition 1.60 or 1.61, wherein the backing layer comprises polyethylene;
1.63. Composition 1, or any of 1.1-1.62, wherein the composition provides whitening of the teeth for a period of time of at least 10 minutes, e.g., 10 minutes to 30 minutes, or 10 minutes to 60 minutes;
1.64. Composition 1, and any of 1.1-1.6 or 1.10-1.54, wherein the composition comprises a whitening agent in addition to the inorganic salt of peroxymonosulfate;
1.65. Composition 1.64, wherein the additional whitening agent comprise a peroxide whitening agent, comprising a peroxide compound;
1.66. Composition 1.64, wherein the peroxide compound is an oxidizing compound comprising a bivalent oxygen-oxygen group;
1.67. Composition 1.65 or 1.66, wherein the peroxide compound is selected from the group consisting of: include hydrogen peroxide, peroxides of alkali and alkaline earth metals, organic peroxy compounds, peroxy acids, pharmaceutically-acceptable salts thereof, and mixtures thereof;
1.68. Composition 1.67, wherein peroxides of alkali and alkaline earth metals are selected from the group consisting of: lithium peroxide, potassium peroxide, sodium peroxide, magnesium peroxide, calcium peroxide, barium peroxide, and mixtures thereof;
1.69. Composition 1.68, wherein the organic peroxy compounds are selected from the group consisting of: carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, and monoperoxyphthalate, and mixtures thereof;
1.70. Composition 1.67, wherein peroxy acids and their salts are selected from the group consisting of: organic peroxy acids such as alkyl peroxy acids, and monoperoxyphthalate and mixtures thereof, as well as inorganic peroxy acid salts such as persulfate, dipersulfate, percarbonate, perphosphate, perborate and persilicate salts of alkali and alkaline earth metals such as lithium, potassium, sodium, magnesium, calcium and barium, and mixtures thereof;
1.71. Composition 1.67, wherein the whitening agent comprises hydrogen peroxide, or urea peroxide, or sodium percarbonate and mixtures thereof;
1.72. Composition 1.67, wherein the peroxide compound comprises hydrogen peroxide;
1.73. Composition 1.67, wherein the whitening agent is a non-peroxide whitening agent;
1.74. Composition 1.73, wherein the non-peroxide whitening agents include is selected from the group consisting of: non-peroxy compounds (e.g., such as chlorine dioxide), chlorites, hypochlorites. And colorants (e.g., titanium dioxide and hydroxyapatite);
1.75. Any foregoing composition further comprising a pH adjustment agent selected from lactic acid, citric acid, hydrochloric acid glycolic acid, sodium hydroxide, potassium chloride, monosodium citrate, disodium citrate, monosodium malate, sodium carbonate; bicarbonates, sesquicarbonates, borates, silicates, monosodium phosphate, tri sodium phosphate, pyrophosphate salts, imidazole, or combinations thereof; e.g., citric acid;
1.76. Any foregoing composition comprising a pH adjustment agent in an amount of 0.01% to 5% (e.g., about 1.5% by wt.);
1.77. Composition 1.73 or 1.74, wherein the pH adjustment agent is citric acid;
1.78. Composition 1.73; or 1.74, wherein the pH adjustment agent is sodium hydroxide, e.g., aqueous sodium hydroxide (e.g., 10% aq. NaOH solution);
1.79. Any foregoing composition wherein the composition, after any pH adjustment, has a pH of about 1 to 7, e.g., about 3 to 7, or about 4 to 6, or about 4 to 5.5, or about 4.5 to 5.5, or about 5 to 6, e.g., or about 4, or about 5, or about 6, or about 7;
1.80. Any foregoing composition further comprising a chelating agent selected from alkali metal stannates (e.g., sodium and potassium stannate) and ethylenediaminetetraacetic acid (EDTA) and its salts;
1.81. Any of the foregoing compositions, wherein the composition is an aqueous slurry;
1.82. Any foregoing composition, wherein the composition comprises greater than 30% by weight of water, e.g., 30 to 90%, or 30 to 80%, or 30 to 70%, or 30 to 60%, or 40 to 90%, or 40 to 80%, or 40 to 70%, or 40 to 60%, or 50 to 90%, or 50 to 80%, or 50 to 70%, or 50 to 60%, or 60 to 90%, or 60 to 80%, or 60 to 70%, or 70 to 90%, or 70 to 80%, or 80 to 90%, by weight.

In a further aspect, the disclosure encompasses a tooth whitening strip (Strip 1) comprising a hydratable adhesive film, e.g., wherein the hydratable adhesive film of the strip comprises or is prepared from an intermediate composition of any of Composition 1 et seq.

For example, in further embodiments, the present disclosure provides (all percentages by weight provided hereinbelow are measured by weight of the hydratable adhesive film of the tooth-whitening strip, e.g., after drying to remove volatile components, such as water or alcohols, and not including any backing layers or protective films):
1.1. Strip 1, wherein the hydratable adhesive film of the strip comprises an inorganic salt of peroxymonosulfate is sodium peroxymonosulfate, potassium peroxymonosulfate, or lithium peroxymonosulfate, or a combination thereof;
1.2. Strip 1 or 1.1, wherein the salt of peroxymonosulfate is potassium peroxymonosulfate;
1.3. Strip 1.2, wherein the potassium peroxymonosulfate is provided as a triple salt of potassium peroxymonosulfate, potassium hydrogen sulfate and potassium sulfate (e.g., Caroat®), optionally wherein the triple salt comprises about 45%-50% by weight of potassium peroxymonosulfate (MPS), e.g., 47% or 49% by weight of potassium peroxymonosulfate;
1.4. Any of the preceding Strips, wherein the hydratable adhesive film of the strip comprises an effective amount of peroxymonosulfate salt which is 0.01% to 10%, by weight, e.g., 0.01% to 5%, 0.05% to 5%, or 0.1% to 5%, or 0.5% to 5%, or 0.5% to 3%, or 0.5% to 2.5%, or 0.5 to 2%, or 0.5 to 1.5%, or 0.75% to 5%, or 0.75% to 4%, or 0.75% to 3%, or 0.75% to 2%, or 0.75 to 1.25%, or 0.8% to 1.2%, or 0.85% to 1.15%, or 0.9% to 1.1%, or 1% to 5%, or 1% to 4%, or 1% to 3%, or 1% to 2%, or 1% to 1.5%, or 2% to 5%, or 2% to 4%, or 2% to 3%, or 3% to 5%, or 3% to 4%, or 2.5% to 3.5%, or about 1%, or about 1.35%, or about 1.5%, or about 1.65%, or about 2%, or about 2.5%, or about 3%, by weight;
1.5. Any of the preceding strips, wherein the hydratable adhesive film of the strip further comprises a second inorganic salt of peroxymonosulfate;
1.6. Any of the preceding strips, wherein the inorganic peroxymonosulfate salt is not particulated (e.g., not granulated);
1.7. Any of the preceding strips, wherein the strip does not comprise hydrogen peroxide;
1.8. Any of the preceding strips, wherein the strip does not comprise any of hydrogen peroxide, urea peroxide, peroxide salts (e.g., sodium peroxide, potassium peroxide, lithium peroxide, calcium peroxide), peroxy acids (e.g., peroxyacetic acid, peroxybenzoic acid, or salts or derivatives thereof), organic peroxides (e.g., urea hydrogen peroxide, glyceryl hydrogen peroxide, peroxy esters, diacyl peroxides, monoperoxyphthalate, or salt thereof), perborate salts, persilicate salts, percarbonate salts, chlorinated oxidizing agents (e.g., hypochlorite salts, chlorite salts, chlorate salts, perchlorate salts, chlorine dioxide);
1.9. Any of the preceding strips, wherein the inorganic salt of peroxymonosulfate is the only oxidizing agent present in the strip;
1.10. Strips 1.9, wherein potassium peroxymonosulfate (e.g., as part of a triple salt mixture) is the only oxidizing agent present in the strip;
1.11. Strip 1, or any of 1.1-1.10, wherein the hydratable adhesive film of the strip comprises one or more water-soluble or water-swellable polymers, including anionic polymers and/or neutral polymers;
1.12. Strip 1.11, wherein the hydratable adhesive film of the strip comprises from 5%-95% by weight of water-soluble or water-swellable polymers or humectants, for example, 5%-90% by weight, or 5%-85% by weight, or 5%-80% by weight, or 5%-70% by weight, or 5%-60% by weight, or 5%-30% by weight, or 10%-95% by weight, or 10%-90% by weight, or 10%-85% by weight, or 10%-80% by weight, or 10%-70% by weight, or 10%-60% by weight, 10-30% by weight, or 10-25% by weight, or 15%-95% by weight, or 15%-90% by weight, or 15%-85% by weight, or 15%-80% by weight, or 15%-70% by weight, or 15%-60% by weight, or 15-25% by weight, or 20%-95% by weight, or 20%-90% by weight, or 20%-85% by weight, or 20%-80% by weight, or 20%-70% by weight, or 30%-95% by weight, or 30%-90% by weight, or 30%-85% by weight, or 30%-80% by weight, or 30%-70% by weight, or 40%-95% by weight, or 40%-90% by weight, or 40%-85% by weight, or 40%-80% by weight, or 40%-70% by weight, or 50%-95% by weight, or 50%-90% by weight, or 50%-85% by weight, or 50%-80% by weight, or 50%-70% by weight, or 60%-95% by weight, or 60%-90% by weight, or 60%-85% by weight, or 60%-80% by weight, or 60%-70% by weight, or 70%-95% by weight, or 70%-85% by weight, or 70%-80% by weight, or 80%-95% by weight, or 80%-90% by weight, or 85%-95% by weight (e.g., about 20% by weight, or about 65% by weight, or about 80% by weight, or about 90% by weight, or about 95% by weight);
1.13. Strip 1, or any of 1.1-1.12, wherein the hydratable adhesive film of the strip comprises one or more humectants selected from: polyethylene glycols (such as PEG-200, PEG-300, PEG-400, PEG-500, PEG-600, PEG-800, PEG-1000, PEG-1600, PEG-2000), polypropylene glycols, polyoxyethylene-polyoxypropylene copolymers (PEG-PPG, including block copolymers, triblock copolymers (poloxamers, such as poloxamer 407), and random copolymers, such as PEG/PPG-116/66 and PEG/PPG-38/8), ethanol, glycerin, propylene glycol, sorbitol, and xylitol;
1.14. Strip 1.13, wherein the hydratable adhesive film of the strip comprises one or more humectants selected from polyethylene glycols, PEG-PPG copolymers, glycerin, and propylene glycol;
1.15. Strip 1.14, wherein the hydratable adhesive film of the strip comprises one or more humectants selected from polyethylene glycols, PEG-PPG copolymers, and glycerin;
1.16. Strip 1.14, wherein the hydratable adhesive film of the strip comprises at least one polyethylene glycol, e.g., selected from PEG-200, PEG-300, PEG-400, PEG-500, PEG-600, PEG-800, PEG-1000, PEG-1600, and PEG-2000;
1.17. Strip 1.16, wherein the hydratable adhesive film of the strip comprises PEG-600;
1.18. Any of Strips 1.13-1.17, wherein the hydratable adhesive film of the strip comprises at least one PEG-PPG copolymer;
1.19. Strip 1.18, wherein the PEG-PPG copolymer is a random copolymer, e.g., PEG/PPG-116/66 or PEG/PPG-38/8;
1.20. Strip 1.18, wherein the PEG-PPG copolymer is a triblock copolymer. i.e., a poloxamer;
1.21. Strip 1.20, wherein the poloxamer is selected from Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, and Poloxamer 407;
1.22. Strip 1.21, wherein the poloxamer is selected from Poloxamer 105 and Poloxamer 407;
1.23. Strip 1.22, wherein the poloxamer is Poloxamer 105;
1.24. Any of Strips 1.13-1.23, wherein the hydratable adhesive film of the strip comprises a total of not more than 30% by weight of said humectants, for example, not more than 24% by weight, not more than 21% by weight, nor more than 18% by weight, not more than 15% by weight, not more than 12% by weight, or not more than 9% by weight, or not more than 7.5% by weight, or not more than 6% by weight, or not more than 3% by weight, or not more than 2% by weight, of said humectants;
1.25. Any of Strips 1.13-1.24, wherein the hydratable adhesive film of the strip comprises from 0.5% by weight to 30% by weight of said humectants, for example, 0.5% to 24% by weight, or 0.5% to 21% by weight, or 0.5 to 18% by weight, or 0.5% to 15% by weight, 0.5% to 12% by weight, or 0.5 to 10% by weight, or 0.5% to 9% by weight, or 2% to 30% by weight, or 2% to 24% by weight, or 2% to 21% by weight, or 2% to 18% by weight, or 2% to 15% by weight, or 2% to 12% by weight, or 2% to 9% by weight, or 4% to 30% by weight, or 4% to 24% by weight, or 4% to 21% by weight, or 4% to 18% by weight, or 4% to 15% by weight, or 4% to 12% by weight, or 4% to 9% by weight, or 6% to 30% by weight, or 6% to 24% by weight, or 6% to 21% by weight, or 6% to 18% by weight, or 6% to 15% by weight, or 6% to 12% by weight, or 8% to 30% by weight, or 8% to 24% by weight, or 8% to 21% by weight, or 8% to 18% by weight, or 10% to 30% by weight, or 10% to 24%, by weight, or 10% to 21% by weight, or 10% to 18% by weight, or about 6%, or about 9% by weight, or about 12% by weight, or about 15% by weight, or about 18% by weight, or about 21%;
1.26. Strip 1.25, wherein the hydratable adhesive film of the strip comprises 1% to 10% by weight of polyethylene glycols, for example, 2% to 10% by weight, or 2% to 8% by weight, or 2% to 7% by weight, or 2% to 6% by weight, or 2% to 5% by weight, or 2% to 4% by weight, or 2% to 3% by weight, or 3% to 10% by weight, or 3% to 8% by weight, or 3% to 7% by weight, or 3% to 6% by weight, or 3% to 5% by weight, or 3% to 4% by weight, or 4% to 10% by weight, or 4% to 8% by weight, or 4% to 7% by weight, or 4% to 6% by weight, or 5% to 10% by weight, or 5% to 8%, by weight, or 5% to 7% by weight, or 5% to 6% by weight, or about 3% by weight, or about 4% by weight, or about 5% by weight, or about 6% by weight, of polyethylene glycols.
1.27. Strip 1.26, wherein the polyethylene glycols comprise PEG-200, PEG-300, PEG-400, PEG-500, PEG-600, PEG-800, or a combination thereof;
1.28. Strip 1.27, wherein the polyethylene glycols comprise PEG-600; 1.29. Any of Strips 1.13-1.28, wherein the strip does not comprise ethanol;
1.30. Any of Strips 1.13-1.29, wherein the strip does not comprise sorbitol or xylitol;
1.31. Any of Strips 1.13-1.30, wherein the strip does not comprise glycerol, or propylene glycol;
1.32. Any of Strips 1.13-1.31, wherein the strip does not comprise propylene glycol;
1.33. Any of Strips 1.13-1.32, wherein the strip does not comprise PEG-PPG copolymers;
1.34. Any of Strips 1.13-1.33, wherein a polyethylene glycol (e.g., PEG-600) is the only humectant;
1.35. Strip 1, or any of 1.1-1.34, wherein the hydratable adhesive film of the strip further comprises one or more polymers selected from polysaccharides (e.g., cellulose derivatives, for example carboxymethyl cellulose, hydroxyethyl cellulose, hydroxypropyl cellulose, hydroxypropyl methyl cellulose, ethyl cellulose, microcrystalline cellulose; or polysaccharide gums, for example xanthan gum, guar gum, or carrageenan gum, pectins, karaya gum); chitosans; dextrans; polyvinyl pyrrolidone (PVP), such as cross-linked PVP; hyaluronic acid and sodium hyaluronates; synthetic anionic polymeric polycarboxylates, such as copolymers of maleic anhydride or acid with another polymerizable ethylenically unsaturated monomer, preferably methyl vinyl ether (e.g., copolymers in a 1:4 to 4:1 ratio of maleic anhydride/acid to methyl vinyl ether); polyphosphonic acids and polyphosphonates (i.e., polyphosphoesters); cross-linked carboxyvinyl copolymers; polyacrylic acid or polyacrylate polymers; polyacrylamides, such as (2-hydroxypropyl)methacrylamide; polyamines; polyvinyl alcohols; polyvinyl pyrrolidine-polyvinyl acetate copolymers (PVP-VA); polyoxazolines, such as poly(2-alkyl-2-oxazolines), e.g., methyl, ethyl, or isopropyl substituted polyoxazolines; and quaternary ammonium polymers;
1.36. Strip 1, or any of 1.1-1.35, wherein the hydratable adhesive film of the strip comprises one or more of: polyethylene glycol polymers (PEG), polyacrylic acid or polyacrylate polymers (PAA), polyvinylpyrrolidone polymers (PVP), polyvinylpyrrolidone-vinyl acetate copolymers (PVP-VA), polyoxazoline polymers (PO), polyoxyethylene-polyoxypropylene (PEG-PPG) block copolymers, and mixtures thereof;
1.37. Strip 1.36, wherein the hydratable adhesive film of the strip comprises at least one of each of: a polyethylene glycol polymer, a polyacrylic acid or polyacrylate polymer, a polyvinylpyrrolidone polymer, a polyvinylpyrrolidone-vinyl acetate copolymer, a polyoxazoline polymer, and a polyoxyethylene-polyoxypropylene copolymer;
1.38. Strip 1, or any of 1.1-1.37, wherein the hydratable adhesive film of the strip comprises at least one of: a cellulose derivative (e.g., carboxymethyl cellulose, hydroxyethyl cellulose, hydroxypropyl cellulose, hydroxypropyl methyl cellulose, or ethyl cellulose), a polyethylene glycol polymer (PEG), a polyacrylic acid or polyacrylate polymer (PAA), a polyvinylpyrrolidone polymer (PVP), a polyvinylpyrrolidone-vinyl acetate copolymer (PVP-VA), a polyoxazoline polymer (PO), a polyoxyethylene-polyoxypropylene (PEG-PPG) block copolymer, or mixtures thereof;
1.39. Strip 1, or any of 1.1-1.38, wherein the hydratable adhesive film of the strip comprises less than 80% by weight of any of said polymers or mixtures of polymers, e.g., 1% to 80%, or 1% to 70%, or 1% to 60%, or 1% to 50%, or 1% to 40%, or 5% to 80%, or 5% to 70%, or 5% to 60%, or 5% to 50%, or 10% to 80%, or 10% to 70%, or 10% to 60%, or 10% to 50%, or 20% to 80%, or 20% to 70%, or 20% to 60%, or 20% to 50%, or 30% to 80%, or 30% to 70%, or 30% to 60%, or 30% to 50%, or 40% to 80%, or 40% to 70%, or 40% to 60%, or 50% to 80%, or 50% to 70%, or 50% to 60%, or 55% to 65%, or 60% to 80% or 60% to70%, or about 50%, or about 60%, or about 70%, or about 80%, by weight;
1.40. Strip 1, or any of 1.1-1.39, wherein the hydratable adhesive film of the strip comprises one or more polyvinylpyrrolidone (PVP) polymers, e.g., linear and/or cross-linked PVP polymers;
1.41. Strip 1.40, wherein the hydratable adhesive film of the strip comprises linear PVP polymers, e.g., one or two linear PVP polymers;
1.42. Strip 1.41, wherein the linear PVP polymers are selected from PVP K90 and PVP K29/32, or a combination thereof;
1.43. Any of Strips 1.40-1.42, wherein the hydratable adhesive film of the strip comprises 40% to 90% by weight of PVP polymers, e.g., 40% to 80% by weight, or 40% to 75% by weight, or 40% to 70% by weight, or 40% to 65% by weight, or 50% to 90% by weight, or 50% to 80% by weight, or 50% to 75% by weight, or 50% to 70% by weight, or 50% to 65% by weight, or 55% to 90% by weight, or 55% to 80% by weight, or 55% to 75% by weight, or 55% to 70% by weight, or 55% to 65%, or 60% to 90% by weight, or 60% to 80% by weight, or 60% to 70% by weight, or 70% to 90% by weight, or 70% to 80% by weight, or 80% to 90% by weight, or 85% to 90% by weight, or about 60% by weight, or about 70% by weight, or about 75% by weight, or about 80% by weight, or about 85% by weight;
1.44. Strip 1, or any of 1.1-1.43, wherein the strip does not comprise a polysaccharide polymer or derivative thereof;
1.45. Strip 1, or any of 1.1-1.43, wherein the strip does not comprise carboxymethyl cellulose, hydroxyethyl cellulose, hydroxypropyl cellulose, hydroxypropyl methyl cellulose, ethyl cellulose;
1.46. Strip 1, or any of 1.1-1.43, wherein the strip does not comprise hydroxypropyl methyl cellulose;
1.47. Strip 1, or any of 1.1-1.43, wherein the strip does not comprise carboxymethyl cellulose;
1.48. Strip 1, or any of 1.1-1.47, wherein the hydratable adhesive film of the strip further comprises a polyphosphate or an organic cyclic polyphosphate, such as an alkali metal pyrophosphate, an alkali metal tripolyphosphate, an alkali metal tetraphosphate, an alkali metal hexametaphosphate, an alkali metal insoluble metaphosphate, an alkali metal phytic acid salt, or a mixture thereof;
1.49. Strip 1.48, wherein the hydratable adhesive film of the strip comprises sodium or potassium pyrophosphate (e.g., tetrasodium or tetrapotassium pyrophosphate and/or disodium or dipotassium pyrophosphate and/or sodium acid pyrophosphate), sodium or potassium tripolyphosphate, sodium or potassium tetraphosphate, sodium or potassium phytic acid salt, or a mixture thereof;
1.50. Strip 1.48 or 1.49, wherein the hydratable adhesive film of the strip comprises from 0.05 to 5% by weight of polyphosphates, e.g., 0.05 to 2%, or 0.05% to 1%, or 0.05% to 0.5% or about 0.2% by weight of polyphosphates;
1.51. Strip 1, or any of 1.1-1.50, wherein the hydratable adhesive film of the strip further comprises a desensitizing agent, e.g., in an amount from 0.1 to 5% by weight, such as potassium nitrate;
1.52. Strip 1, or any of 1.1-1.51, wherein the hydratable adhesive film of the strip further comprises an enamel strengthening agent, e.g., in an amount from 0.1 to 5% by weight, such as zinc phosphate;
1.53. Strip 1 or any of 1.1-1.52, wherein the hydratable adhesive film of the strip further comprises one or more of flavors and sweeteners;
1.54. Strip 1, or any of 1.1-1.53, wherein the hydratable adhesive film of the strip consists of PVP, potassium peroxymonosulfate triple salt (e.g., Caroat®), and one or more of polyethylene glycol (e.g., PEG-600), PEG-PPG copolymer (e.g., Poloxamer 105), glycerin, and optionally water;
1.55. Strip 1, or any of 1.1-1.53, wherein the hydratable adhesive film of the strip consists of water, PVP, potassium peroxymonosulfate triple salt (e.g., Caroat®), and polyethylene glycol (e.g., PEG-600);
1.56. Strip 1 or any of 1.1-1.55, wherein the hydratable adhesive film of the strip comprises or is prepared from an intermediate composition of any of Composition 1 et seq.;
1.57. Strip 1.56, wherein the intermediate composition is an aqueous slurry that comprises or consists of:

| | Amount by |
|---|---|
| | wt. % of the |
| Component | slurry |
| MPS (e.g., provided as | 0.5-2% (e.g., 1%) |
| Caroat ®, 45% MPS) | |
| Water | q.s. (e.g., about 75%) |
| PVP | 10-20% |
| PEG 600 | 3-8% |
| Chelating agent | 0.1-1% |
| pH adjusting agent | 0.5-3% |
| Alkali metal pyrophosphate | 0.1-1%; |

1.58. Strip 1.56, wherein the intermediate composition is an aqueous slurry that comprises or consists of:

| | Amount by |
|---|---|
| | wt. % of the |
| Component | slurry |
| MPS (e.g., provided as | 0.3-1.5% |
| Caroat ®, 45% MPS) | (e.g., 0.6%) |
| Water | q.s. (e.g., about 60-80%) |
| PVP | 15-30% (e.g., 25% |
| PEG 600 | 1-5% (e.g., 2% |
| pH adjusting agent (e.g. NaOH) | 0.5-3% |

or

| | Amount by |
|---|---|
| | wt. % of the |
| Component | slurry |
| MPS (e.g., provided as | 0.5-3% (e.g., 0.9%) |
| Caroat ®, 45% MPS) | |
| Water | q.s. (e.g., about 60-80%) |
| PVP (e.g., PVP K90 and/or | 15-30% |
| PVP K29/32) | (e.g., 25%) |
| PEG (e.g., PEG 600) | 0.5-5% (e.g., 2%) |
| pH adjusting agent (e.g. NaOH) | 0.5-3%; |

1.59. Strip 1, or any of 1.1-1.58, wherein the hydratable adhesive film of the strip comprises 40-90% by weight of PVP, 1-6% by weight of potassium peroxymonosulfate triple salt (e.g., Caroat®; or about 0.5-2% by weight of potassium peroxymonosulfate (MPS)), and 2-20% of one or more of polyethylene glycol (e.g., PEG-600), PEG-PPG copolymer (e.g., Poloxamer 105), glycerin, and water;
1.60. Strip 1, or any of 1.1-1.58, wherein the hydratable adhesive film of the strip comprises 40-90% by weight of PVP, 1-6% by weight of potassium peroxymonosulfate triple salt (e.g., Caroat®; or about 0.5-2% by weight of potassium peroxymonosulfate (MPS)), and 2-10% of one or more of polyethylene glycol (e.g., PEG-600), PEG-PPG copolymer (e.g., Poloxamer 105), and glycerin; and 0-15% water;
1.61. Strip 1, or any of 1.1-1.58, wherein the hydratable adhesive film of the strip comprises 40-90% by weight of PVP, 1-6% by weight of potassium peroxymonosulfate triple salt (e.g., Caroat®; or about 0.5-2% by weight of potassium peroxymonosulfate (MPS)), and 2-10% of polyethylene glycol (e.g., PEG-600); and 0-15% water;
1.62. Strip 1, or any of 1.1-1.58, wherein the hydratable adhesive film of the strip comprises 40-90% by weight of PVP, 1-6% by weight of potassium peroxymonosulfate triple salt (e.g., Caroat®; or about 0.5-2% by weight of potassium peroxymonosulfate (MPS)), and 2-10% of one or more of polyethylene glycol (e.g., PEG-600), PEG-PPG copolymer (e.g., Poloxamer 105), and glycerin; and 0-15% water;
1.63. Strip 1, or any of 1.1-1.62, wherein the hydratable adhesive film of the strip comprises 0-20% water (i.e., after drying), for example, 0-16%, or 0-14%, or 0-12%, or 0-10%, or 0-5%, or 5-15%, or 8-12%, or about 11%, by weight of the strip. 1.64. Strip 1, or any of 1.1-1.63, wherein the hydratable adhesive film of the strip comprises or consists of:

| | Amount by |
|---|---|
| Component | wt. % |
| MPS (e.g., provided as | 1-6% (e.g., 0.5-2%) |
| Caroat ®, 45% MPS) | |
| Water | q.s. (e.g., about 0-15%) |
| PVP (e.g., PVP K90 and/or | 40-90% |
| PVP K29/32) | (e.g., 70-90%) |
| PEG (e.g., PEG 600), PEG- | 2-10% (e.g., 4-8%) |
| PPG copolymer, and/or glycerin | |
| pH adjusting agent (e.g. | 1-10% |
| NaOH) | (e.g., 5%) |

or

| | Amount by |
|---|---|
| Component | wt. % |
| MPS (e.g., provided as | 0.5-3% (e.g., 0.9-1.1%) |
| Caroat ®, 45% MPS) | |
| Water | q.s. (e.g., about 0-15%) |
| PVP (e.g., PVP K90 and/or | 50-90% |
| PVP K29/32) | (e.g., 75-85%) |
| PEG (e.g., PEG 600) | 2-10% (e.g., 4-8%) |
| pH adjusting agent (e.g. | 1- 0% |
| NaOH) | (e.g., 5%); |

1.65. Strip 1, or any of 1.1-1.64, wherein the strip further comprises an inert paper or plastic backing layer to which the hydratable adhesive film is adhered or affixed, and/or an inert removable paper or plastic protective film adhered to the hydratable adhesive film, optionally, wherein the protective film and/or the backing strip are comprised of wax paper, coated paper, treated paper, polyethylene, polystyrene, polypropylene, polyvinyl chloride, polyvinyl acetate, polymethyl methacrylate, polytetrafluoroethylene, polyesters (e.g., polyethylene terephthalate), polyimides, polycarbonates, or any combination thereof;
1.66. Strip 1, or any of 1.1-1.65, wherein the intermediate strip is prepared as an aqueous suspension or aqueous/alcoholic suspension and subsequently dried onto a backing layer of the tooth whitening strip;
1.67. Strip 1, or any of 1.1-1.66, wherein the strip is prepared from any of intermediate Composition 1 et seq., and wherein the intermediate composition is prepared as an aqueous suspension or aqueous/alcoholic suspension and subsequently dried onto a backing layer of the tooth whitening strip, and wherein the strip further comprises an inert removable paper or plastic protective film and/or an affixed inert paper or plastic backing strip and wherein said protective film or backing strip is adhered to the hydratable adhesive film, optionally, wherein the protective film and/or the backing strip are comprised of wax paper, coated paper, treated paper, polyethylene, polystyrene, polypropylene, polyvinyl chloride, polyvinyl acetate, polymethyl methacrylate, polytetrafluoroethylene, polyesters (e.g., polyethylene terephthalate), polyimides, polycarbonates, or any combination thereof;
1.68. Any of Strips 1.65 to 1.67, wherein the protective film and/or the backing strip comprises polypropylene, polyethylene, polyethylene terephthalate, and/or polystyrene;
1.69. Strip 1.67, wherein the backing layer comprises polyethylene and/or the protective film comprises polyethylene or polypropylene;
1.70. Strip 1, or any of 1.1-1.69, wherein the strip provides whitening of the teeth for a period of time of at least 10 minutes, e.g., 10 minutes to 30 minutes, or 10 minutes to 60 minutes;
1.71. Strip 1, and any of 1.1-1.6 or 1.10-1.70, wherein the strip comprises a whitening agent in addition to the inorganic salt of peroxymonosulfate;
1.72. Strip 1.71, wherein the additional whitening agent comprise a peroxide whitening agent, comprising a peroxide compound;
1.73. Strip 1.71, wherein the peroxide compound is an oxidizing compound comprising a bivalent oxygen-oxygen group;
1.74. Strip 1.72 or 1.73, wherein the peroxide compound is selected from the group consisting of: include hydrogen peroxide, peroxides of alkali and alkaline earth metals, organic peroxy compounds, peroxy acids, pharmaceutically-acceptable salts thereof, and mixtures thereof;
1.75. Strip 1.74, wherein peroxides of alkali and alkaline earth metals are selected from the group consisting of: lithium peroxide, potassium peroxide, sodium peroxide, magnesium peroxide, calcium peroxide, barium peroxide, and mixtures thereof;
1.76. Strip 1.75, wherein the organic peroxy compounds are selected from the group consisting of: carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, and monoperoxyphthalate, and mixtures thereof;
1.77. Strip 1.75, wherein peroxy acids and their salts are selected from the group consisting of: organic peroxy acids such as alkyl peroxy acids, and monoperoxyphthalate and mixtures thereof, as well as inorganic peroxy acid salts such as persulfate, dipersulfate, percarbonate, perphosphate, perborate and persilicate salts of alkali and alkaline earth metals such as lithium, potassium, sodium, magnesium, calcium and barium, and mixtures thereof;
1.78. Strip 1.75, wherein the whitening agent comprises hydrogen peroxide, or urea peroxide, or sodium percarbonate and mixtures thereof;
1.79. Strip 1.75, wherein the peroxide compound comprises hydrogen peroxide;
1.80. Strip 1.75, wherein the whitening agent is a non-peroxide whitening agent;
1.81. Strip 1.75, wherein the non-peroxide whitening agents include is selected from the group consisting of: non-peroxy compounds (e.g., such as chlorine dioxide), chlorites, hypochlorites. And colorants (e.g., titanium dioxide and hydroxyapatite);
1.82. Any foregoing strip further comprising a pH adjustment agent selected from lactic acid, citric acid, hydrochloric acid, glycolic acid, sodium hydroxide, potassium chloride, monosodium citrate, disodium citrate, monosodium malate, sodium carbonate, bicarbonates, sesquicarbonates, borates, silicates, monosodium phosphate, trisodium phosphate, pyrophosphate salts, imidazole, or combinations thereof; e.g., citric acid;
1.83. Any foregoing strip comprising a pH adjustment agent in an amount of 0.01% to 5% (e.g., about 1.5% by wt.);
1.84. Strip 1.82 or 1.83, wherein the pH adjustment agent is citric acid;
1.85. Strip 1.82 or 1.83 wherein the pH adjustment agent is sodium hydroxide, e.g., aqueous sodium hydroxide (e.g., 10% aq. NaOH solution);
1.86. Any foregoing strip wherein the strip has a pH of about 1 to 7, e.g., about 3 to 7, or about 4 to 6, or about 4 to 5.5, or about 4.5 to 5.5, or about 5 to 6, e.g., about 4, about 5, or about 6, measured before drying of the strip or after hydrating the strip;
1.87. Any foregoing strip further comprising a chelating agent selected from alkali metal stannates (e.g., sodium and potassium stannate) and ethylenediaminetetraacetic acid (EDTA) and its salts;
1.88. Any of the foregoing strips, wherein the intermediate strip is an aqueous slurry;
1.89. Any of the foregoing strips—wherein the strip is free, or substantially free (e.g., less than 0.005 wt. %) of water.
1.90. Any foregoing strip, wherein the strip is free, or substantially free, of glycerin, sorbitol, xylitol, propylene glycol, and/or ethanol, e.g., less than 0.5% by weight of each, or less 0.1% by weight of each.
1.91. Use of any of the preceding strips in a method for whitening teeth.
1.92. A method of whitening teeth, wherein the method comprises applying a strip of any of any of Strip 1 or 1.1-1.90 to one or more teeth.

As used herein, the term “strip” refers to a solid, pliable, adherent material which comprises a single-layer hydratable film comprising an inorganic peroxymonosulfate salt entrained or dispersed in a polymer matrix, and optionally a backing for the front and/or rear surfaces of the film. The film, after wetting with water or saliva or assisting solvent, and upon adherence to the surface of the teeth, provides an aqueous vehicle which permits the inorganic peroxymonosulfate salt to diffuse towards the surface of the teeth to provide an oxidative whitening effect on the teeth.

As used herein, the term “oral care composition” means the total composition that is delivered to the oral surfaces. The composition is further defined as a product which, during the normal course of usage, is not, the purposes of systemic administration of particular therapeutic agents, intentionally swallowed but is rather retained in the oral cavity for a time sufficient to contact substantially all of the dental surfaces and/or oral tissues for the purposes of oral activity. Examples of such compositions include, but are not limited to, toothpaste or a dentifrice, a mouthwash or a mouth rinse, a topical oral gel, a denture cleanser, sprays, toothpaste powders, tablets, mousse, foam, lozenge, ribbon, chewing gum and the like.

As used herein, “slurry” or “aqueous slurry” refers to a semiliquid mixture comprising water and at least one other ingredient suspended in the mixture. In one aspect, the intermediate composition of the disclosure is an aqueous slurry which is then incorporated within a tooth whitening strip comprising a hydratable adhesive film. In one aspect, the slurry is incorporated within the strip following a drying process which can remove water and other volatile ingredients used as solvents.

In one embodiment, the peroxymonosulfate is potassium peroxymonosulfate (also known as MPS, potassium monopersulfate). The potassium peroxymonosulfate (an example of which is Caroat ° or Oxone®, an oxidizing agent) may be combined to form or exist as a triple salt of potassium peroxymonosulfate, potassium hydrogen sulfate and potassium sulfate (2KHSO₅·KHSO₄·K₂SO₄).

Potassium peroxymonosulfate has limited stability in aqueous solutions and can be stabilized by other common toothpaste ingredients. Therefore, contact with water during processing and storage should be avoided or minimized. The strip is preferably packaged in a moisture free environment. Preferably, strips are individually packaged and sealed into unit dose packages. The strips are typically stored in an air tight, moisture-proof package, e.g., sachets, sealed metal foil pouches, blister packs, and desiccant capped tubes. Useful packaging materials include polymeric packaging (e.g., polyethylene and polypropylene), metal foil packaging (e.g., aluminum), and combinations thereof.

The resulting strips that incorporate the intermediate composition of the present disclosure contain no water or have a low water content. As used herein, the term “low water content” means the total concentration of water, including any free water and all water contained in any ingredients. In various embodiments of the composition, the amount of water in the final resulting strip is in an amount of less than 4% by weight, or less than 3% by weight, or less than 2% by weight, or less than 1% by weight, or less than 0.5% by weight, or less than 0.1%, or about 0.0001% to about 4% by weight, or about 0.0001% to about 0.5% by weight or about 0.0001% to about 0.1% by weight.

In some embodiments, the compositions of the present disclosure contain a buffering agent. Examples of buffering agents include anhydrous carbonates such as sodium carbonate, sesquicarbonates, bicarbonates such as sodium bicarbonate, silicates, bisulfates, phosphates such as monopotassium phosphate and dipotassium phosphate, citrates, pyrophosphates (sodium and potassium salts) and combinations thereof. The amount of buffering agent is sufficient to provide a pH of about 3 to about 9, preferably about 6 to about 8, and more preferably at about 7, when the strip is hydrated. Typical amounts of buffering agent are about 0.1% to about 5%, in one embodiment about 1% to about 3%, in another embodiment about 0.5% to about 1%, by weight of the total composition.

The compositions and strips of the present disclosure optionally contain a binder, preferably a polymeric binder, which is compatible with an oxidizing agent, which adds bulk to the hydratable film, and assists in holding the components of the film together in the form of a strip. Examples of suitable polymeric binders include, e.g., starches, natural gums, (e.g., xanthan gum), cellulose gums, microcrystalline cellulose, maltodextrins, methylcellulose, cellulose ethers, sodium carboxymethylcellulose, ethylcellulose, gelatin, polyethylene glycol, polyvinylpyrrolidone, pectins, alginates, polyacrylamides, polyvinyloxazolidone, polyvinyl alcohols and mixtures thereof. The binder can also comprise one or more non-polymeric binders such as dextrose, lactose, sucrose, sorbitol, mannitol, xylitol, and the like. Typically, the binder is present in the composition in an amount of about 0.1% by weight to about 80% by weight, about 1% by weight to about 10% by weight, or about 1% by weight to about 5% by weight.

In some embodiments, the intermediate composition comprises a poloxamer, which is a polyoxyethylene-polyoxypropylene triblock copolymer. For example, suitable poloxamers may include one or more of Pluronic® L35, Pluronic® L43, Pluronic® L64, Pluronic® L10, Pluronic® L44, Pluronic® L62, Pluronic® 10R5, Pluronic® 17R4, Pluronic® L25R4, Pluronic® P84, Pluronic® P65, Pluronic® PI 04, and Pluronic® PI 05. Pluronic® brand dispersants are commercially available from BASF, Florham Park, NJ.

In some embodiments, the intermediate composition of the present disclosure may comprise a cross-linked polyvinylpyrrolidone, also known as poly-N-vinyl-poly-2-pyrrolidone, and commonly abbreviated to cross-linked “PVP.” PVP generally refers to a polymer containing vinylpyrrolidone (also referred to as N-vinylpyrrolidone, N-vinyl-2-pyrrolidinone and N-vinyl-2-pyrrolidinone) as a monomeric unit. The monomeric unit may include a polar imide group, four non-polar methylene groups, and a non-polar methane group. Cross linked PVP includes those commercially available as KOLLIDON® and LUVICROSS®, marketed by BASF, Mount Olive, N.J., USA; and POLYPLASDO E® INF-10, marketed by, Ashland, Covington, Kentucky, USA.

In some embodiments, a PVP copolymer is employed, such as a polyvinylpyrrolidone-vinyl acetate copolymer (PVP-VA) (e.g., commercially available as Plasdone S-630 from Ashland Inc.), a polyvinyl pyrrolidone-co-polyvinyl butyrate copolymer (PVP-VB), or a polyvinyl pyrrolidone-co-polyvinyl propionate copolymer (PVP-VP), or mixtures thereof.

The intermediate composition of the present disclosure can optionally contain whitening agents in addition to the peroxymonosulfate salt. Whitening agents are generally materials which are effective to provide whitening of a tooth surface to which it is applied, and include agents such as hydrogen peroxide and urea peroxide. In various embodiments, the compositions of the present disclosure may optionally comprise a peroxide whitening agent, comprising a peroxide compound. A peroxide compound is an oxidizing compound comprising a bivalent oxygen-oxygen group. Peroxide compounds include peroxides and hydroperoxides, such as hydrogen peroxide, peroxides of alkali and alkaline earth metals, organic peroxy compounds, peroxy acids, pharmaceutically-acceptable salts thereof, and mixtures thereof. Peroxides of alkali and alkaline earth metals include lithium peroxide, potassium peroxide, sodium peroxide, magnesium peroxide, calcium peroxide, barium peroxide, and mixtures thereof. Organic peroxy compounds include carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, and monoperoxyphthalate, and mixtures thereof. Peroxy acids and their salts include organic peroxy acids such as alkyl peroxy acids, and monoperoxyphthalate and mixtures thereof, as well as inorganic peroxy acid salts such as persulfate, dipersulfate, percarbonate, perphosphate, perborate and persilicate salts of alkali and alkaline earth metals such as lithium, potassium, sodium, magnesium, calcium, and barium, and mixtures thereof. In various embodiments, the peroxide compound comprises hydrogen peroxide, urea peroxide, sodium percarbonate and mixtures thereof. In some embodiments, the peroxide compound comprises hydrogen peroxide. In some embodiments, the peroxide compound consists essentially of hydrogen peroxide. In some embodiments a non-peroxide whitening agent may be provided. Whitening agents among those useful herein include non-peroxy compounds, such as chlorine dioxide, chlorites and hypochlorites. Chlorites and hypochlorites include those of alkali and alkaline earth metals such as lithium, potassium, sodium, magnesium, calcium, and barium. Non-peroxide whitening agents also include colorants, such as titanium dioxide and hydroxyapatite. One or more additional whitening agents are optionally present in a tooth-whitening effective total amount. In some embodiments the compositions additionally comprise an activator, e.g., tetraacetylethylenediamine. In some embodiments, the strips of the present invention are free of all of the above enumerated additional whitening agents.

The intermediate composition of the present disclosure optionally can also include other ingredients, e.g., flavor agents; fillers; surfactants; preservatives, e.g., sodium benzoate and potassium sorbate; color agents including, e.g., dyes and pigments; and sweeteners.

Examples of the surfactant that can be used are sodium lauryl sulfate, sorbitan fatty acid ester, polyoxyethylene (20) sorbitan monooleate (Polysorbate 80 or Tween 80), polyethylene glycol fatty acid ester, polyoxyethylene sorbitan fatty acid ester, polyoxyethylene alkyl ether, polyoxyethylene polyoxypropylene alkyl ether, polyoxyethylene polyoxypropylene block copolymer, polyoxyethylene alkyl phenyl ether, polyoxyethylene castor oil, polyoxyethylene hydrogenated castor oil, polyoxyethylene sorbitol fatty acid ester and polyoxyethylene glycerol fatty acid ester. In the present disclosure, each of them may be used solely or two or more thereof may be used jointly. Typical amounts of surfactant are about 0.1% to about 3%, in one embodiment about 0.1% to about 2%, in another embodiment about 0.1% to about 1%, by weight of the total composition.

Examples of the filler are crystalline cellulose, ethylcellulose, dextrin, various kinds of cyclodextrin (α-cyclodextrin, β-cyclodextrin and γ-cyclodextrin), sodium sulfate, as well as derivatives thereof and pullulan.

Useful flavor agents include natural and synthetic flavoring sources including, e.g., volatile oils, synthetic flavor oils, flavoring aromatics, oils, liquids, oleoresins, and extracts derived from plants, leaves, flowers, fruits, stems, and combinations thereof. Suitable flavor agents include, e.g., citric oils, e.g., lemon, orange, grape, lime and grapefruit, fruit essences including, e.g., apple, pear, peach, grape, strawberry, raspberry, cherry, plum, pineapple, apricot, and other fruit flavors. Other useful flavor agents include, e.g., aldehydes and esters (e.g., benzaldehyde (cherry, almond)), citral, i.e., alpha-citral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), 2-dodecenal (citrus, mandarin) and mixtures thereof.

Suitable coloring agents include, e.g., food, drug and cosmetic (FD&C) colors including, e.g., dyes, lakes, and certain natural and derived colorants. Useful lakes include dyes absorbed on aluminum hydroxide and other suitable carriers.

Suitable sweetening agents include stevia, sugars such as sucrose, glucose, invert sugar, fructose, ribose, tagalose, sucralose, maltitol, erythritol, xylitol, and mixtures thereof, saccharin and its various salts (e.g., sodium and calcium salt of saccharin), cyclamic acid and its various salts, dipeptide sweeteners (e.g., aspartame), acesulfame potassium, dihydrochalcone, glycyrrhizin, and sugar alcohols including, e.g., sorbitol, sorbitol syrup, mannitol and xylitol, and combinations thereof.

It is understood that while general attributes of each of the above categories of materials may differ, there may be some common attributes and any given material may serve multiple purposes within two or more of such categories of materials. All of the ingredients in the compositions may have functions in addition to their primary function, and may contribute to the overall properties of the composition, including its stability, efficacy, consistency, mouthfeel, taste, odor and so forth. For example, a binder may also function as a disintegrating agent and vice versa.

In a second aspect, the present disclosure provides a method for manufacturing a tooth whitening strip comprising the steps of (a) applying any of Composition 1 et seq (e.g., an aqueous slurry comprising MPS), to a backing layer, and (b) drying the Composition 1, et seq to the backing layer to form the hydratable adhesive layer of the strip (e.g., by removing water and other volatile ingredients, e.g., where the ingredients are used as solvents).

In other embodiments, the present disclosure provides for use of any of Composition 1 et seq., or any other embodiments thereof, in the manufacture of a tooth whitening strip comprising a hydratable adhesive film, wherein the tooth whitening strip is further administered to a subject in need thereof as part of a method for the whitening of the teeth.

### EXAMPLES

Exemplary embodiments of the present disclosure will be illustrated by reference to the following examples, which are included to exemplify, but not to limit the scope of the present invention.

In the examples and elsewhere in the description of the invention, chemical symbols and terminology have their usual and customary meanings. Temperatures are in degrees Celsius unless otherwise indicated. The amounts of the components are in weight percent based on the standard described; if no other standard is described then the total weight of the composition is to be inferred. Various names of chemical components include those listed in the CTFA International Cosmetic Ingredient Dictionary (Cosmetics, Toiletry and Fragrance Association, Inc., 7^{th} ed. 1997).

### Example 1

Different MPS slurries are prepared and applied to polystyrene petri dishes to form the dry strips following a drying process. After the dry strips are formed, the active oxygen in the strips is monitored at initial and 8 days under room-temperature storage.

The formulas of MPS slurries are listed in Table 2.

TABLE 1
Formulas of MPS Slurries Containing Different Humectant Species and Levels:

| | Formula | Formula | Formula | Formula | Formula | Formula | Formula |
|---|---|---|---|---|---|---|---|
| | 1 | 2 | 3 | 4 | 5 | 6 | 7 |
| Component | Wt. % | Wt. % | Wt. % | Wt. % | Wt.% | Wt. % | Wt. % |
| Caroat (45% MPS) | 2.2 | 2.2 | 2.2 | 2.2 | 2.2 | 2.2 | 2.2 |
| Water | 71.2 | 69.2 | 64.7 | 69.2 | 64.7 | 69.2 | 64.7 |
| PVP | 25 | 25 | 25 | 25 | 25 | 25 | 25 |
| Glycerin | 0 | 2 | 6.5 | — | — | — | — |
| PEG 600 | — | — | — | 2 | 6.5 | — | — |
| Polyoxypropylene- | — | — | — | — | — | 2 | 6.5 |
| Polyoxyethylene | | | | | | | |
| Copolymer | | | | | | | |
| (EO−- PO | | | | | | | |
| polymers) | | | | | | | |
| 10% NaOH | 1.6 | 1.6 | 1.6 | 1.6 | 1.6 | 1.6 | 1.6 |

The retention rate of active oxygen in the dry strip is calculated and used to evaluate the MPS stability.

$$Retention Rate of Active Oxygen ( \% ) = \frac{Active Oxygen ( aged )}{Active Oxygen ( initial )} \times 100 \%$$

Generally speaking, the level of humectant added in the MPS slurry can impact the stability of MPS in the dry strip. Comparing the result of 0% humectant, 2% humectant and 6.5% humectant, the higher level of humectant that is added to the MPS slurry, the lower retention rate of active oxygen in the dry strip that is achieved. However, the humectant is believed to be necessary in the strip to provide certain properties, such as stretching, which are important for practical application of the strip.

The humectant type also makes an impact to the MPS stability in the dry strip when its addition level is relatively high. When the humectant level is 2% in slurry, the difference of MPS stability in the strip is minor among Glycerin, PEG 600 and EO-PO Copolymer. However, when the humectant level is 6.5% in slurry, the difference of MPS stability in the strip becomes larger among three humectant types. And, without being bound by theory, the degree of impact appears to be consistent with the number of hydroxyl groups in the humectant's molecular structure: Glycerin>PEG 600>EO-PO polymer. These results are listed in Table 2 below.

The Retention Rate of Active Oxygen in MPS strip with Different Humectant types and levels after eight days at room temperature

TABLE 2

| | Formula 1 | Formula 2 | Formula 3 | Formula 4 | Formula 5 | Formula 6 | Formula 7 |
|---|---|---|---|---|---|---|---|
| Humectant | 0% | 2% | 6.5% | 2% | 6.5% | 2% | 6.5% |
| in MPS | | | | | | | |
| slurry | | | | | | | |
| Retention | 96.44 | 89.19 | 76.51 | 91.31 | 84.65 | 89.84 | 88.15 |
| Rate of | | | | | | | |
| Active | | | | | | | |
| Oxygen in | | | | | | | |
| MPS strip, % | | | | | | | |

When increasing the humectant level from 2% to 6.5%, this difference is largely amplified in the dry strip after water is evaporated, as shown in Table 3. After drying, slurry with 2% humectant can be converted to dry strips with 6% residual humectant. This level of residual humectant is relatively safe to MPS. However, slurry with 6.5% humectant is converted to dry strips with 26% residual humectant. This level of residual humectant in dry strips is believed to be too elevated for incorporation of MPS.

TABLE 3
Formulas of MPS Strip Slurries Containing Different Humectant Species and
Levels:

| | Water content | Humectant dosage |
|---|---|---|
| Slurry Formula | analyzed in dry film, % | calculated in dry film, % |
| Slurry Formula 2 | 11.43% | ≈6% |
| (2% Glycerin) | | |
| Slurry Formula 3 | 10.08% | ≈26% |
| (6.5% Glycerin) | | |

### Example 2

Process for Making an MPS Slurry

In certain aspects, making a dry or wet oral strip usually starts with making a slurry, followed by a drying process which removes water and other volatile ingredients used as solvents. It is possible for whitening actives that have limited stability in the slurry could still retain their effectiveness once the subsequent drying process is complete. Accordingly, it is beneficial to develop a suitable working slurry which allows for MPS to be stabilized in the final dry strips for an effective tooth whitening treatment.

Film forming agents are critical for making oral strips and can be made using water soluble polymers such as polyvinylpyrrolidone (PVP), polyvinyl alcohol (PVA), hydroxypropyl cellulose (HPC), Hydroxyethyl cellulose (HEC), methyl cellulose (MC), hydroxypropyl methylcellulose (HPEC), for example. The solvents used in the slurry are believed to possibly present issues when combined with MPS. Glycerin is believed to help prevent the film from becoming overly dry and provides the elasticity to the strips. However, glycerin is believed to impact the stability of MPS, which can be dependent on the concentration of glycerin.

Table 4 lists the results of the stability of MPS, up to two days, in a few experimental compositions containing glycerin and MPS. In these experimental formulas, Caroat (United Initiator) is used as the source of potassium monopersulfate compound and is dosed at 2.2 wt % (45% MPS) to deliver 1% MPS with a theoretical active oxygen level (AO) of 0.105%. The AO levels in the formulas are monitored using the iodine titration method once per day over two days. As seen in Table 4, below, the stability of MPS in the experimental formulas decreases rapidly over a short period of time in a slurry containing glycerin.

TABLE 4
Experimental Formulas with Glycerin and MPS

| | Formula #8 | | Formula #9 | | Formula #10 | | Formula #11 | |
|---|---|---|---|---|---|---|---|---|
| Formula | Glycerin | 97.8% | Glycerin | 57.8% | Glycerin | 30% | Glycerin | 2% |
| | Caroat * | 2.2% | Caroat* | 2.2% | Caroat* | 2.2% | Caroat* | 2.2% |
| | Total | 100.0% | Ethanol | 7.5% | L35 | 37.8% | Water | 55.8% |
| | | | Water | 7.5% | Water | 10% | PVP | 15% |
| | | | PVP | 15% | PVP | 15% | HPC | 5% |
| | | | HPC | 10% | HPC | 5% | Total | 100.0% |
| | | | Total | 100% | Total | 100% | | |
| Active | Initial | 0.088% | Initial | 0.103% | Initial | 0.102% | Initial | 0.112% |
| Oxygen | Day 2 | 0.07% | Day 1 | 0.068% | Day 1 | 0.0857% | Day 1 | 0.0824% |
| | | | | | Day 2 | 0.0784% | Day 2 | 0.0754% |

*Caroat (45% MPS)

Glycerin has an immediate negative impact on MPS stability, as seen in Formula #8 containing only glycerin and MPS. The initial active oxygen (“AO”) level, measured only a few hours after the formula was made, was 0.088%, indicating nearly 16% loss in a very short period of time. After two days, the AO level decreases to 0.07% indicating an approximate 34% loss of MPS. When glycerin content is lowered down to the 50-60% range e.g., 57.8% in Formula #9, there is no significant immediate loss of MPS as the AO level is 0.103%.

There is still a large loss of about 35% MPS after one day. Further reducing glycerin content to 30%, as in Formula #10, appears reduce the degradation of MPS over time. After one day, the experimental formula still has 82% MPS, and after two days, more than 75% MPS remained. The total loss of AO from one and two days, 18% and 25% loss respectively, is less than other experimental formulas. Similar results are seen in formula #11 where water was used as the main solvent and the glycerin content was significantly reduced to 2%. It showed that formulas with glycerin at 30% or less were capable of retaining MPS up to 75% over two days. Additionally, neither water nor L35 negatively impacted MPS stability. The study demonstrates that Formula #10 and #11 have potential for use as an MPS strip slurry base.

When MPS is dissolved in water, it makes the solution very acidic (pH<3). The experimental Formulas: #9, #10 and #11 are measured to have a pH around 2-3. Making strips using these formulas could cause the pH near the enamel to drop after being wetted in the mouth despite the buffering capacity of saliva. Further studies are carried out to determine if MPS could be stabilized in films with a higher initial pH in the slurry. The composition and corresponding test results are listed in Table 5.

TABLE 5
Experimental Formula with Higher pH

| | Formula #12 | | Formula #13 | |
|---|---|---|---|---|
| Formula | Premix A | | Premix A | |
| Composition | Caroat | 2.2% | Caroat | 2.2% |
| | Water | 17.8% | Water | 17.8% |
| | Premix B | | Premix B | |
| | Glycerin | 2% | Glycerin | 2% |
| | Water | 53% | Water | 53% |
| | PVP | 20% | PVP | 20% |
| | HPC | 5% | HPC | 5% |
| | Total | 100.0% | Total | 100.0% |

| | | |
|---|---|---|
| | 10% NaOH Soln. | 10% NaOH Soln. |
| | 500 ul | 400 ul |
| pH | 6.69 | 6.18 |
| AO of the slurry, | Initial 0.049% | Initial 0.0557% |
| % | | |
| AO of the dry | Initial 0.12% | Initial 0.19% |
| film, % | Day 5 0.095% | Day 5 0.214% |

The procedure to make a higher pH experimental formula is as follows:
1. Premix A: Make a 11% MPS water solution by dissolving Caroat into DI water
2. Premix B: Make a glycerin/water base with film forming agents
3. Mix the above two premixtures well
4. Add 10% NaOH into the formula to adjust pH
5. Test initial AO of the experimental formula slurry
6. Spread a small quantity of experiment formula into a thin layer in a petri dish and leave it to open air overnight to turn to a dry film naturally
7. Test AO of the dry film the next day and 5 days later

The results demonstrate that using a NaOH water solution can effectively raise the pH of the experimental formulas to above 6. The addition of the NaOH resulted in some degree of degradation of MPS, for example, the active oxygen was reduced to half of the theoretical formulated level of 0.105% to nearly 0.05% in both formulas. However, after the experimental formula was converted into a film by air drying, the AO level increased. Without being bound by theory, it is believed that this increase is possibly due to the loss of water from the drying process. Once the film is dry, MPS is found to be stable over the course of five days as the AO of the films did not decrease. Without being bound by theory, the increased AO level over time is likely due to further water evaporation which further concentrates the MPS in the film.

### Example 3

A representative Formula of MPS Slurry is provided as follows:

TABLE 6
Formula of MPS Slurry

| | Amount by |
|---|---|
| Component | wt. % |
| Caroat (45% MPS) | 2.2 |
| Water | q.s. (e.g., about 75%) |
| PVP | 10-20% |
| PEG 600 | 3-8% |
| EDTA | 0.1-1% |
| 10% NaOH | 0.5-3% |
| Alkali metal pyrophosphate | 0.1-1% |

### Example 4

A MPS wet slurry is applied to different packaging materials to form the dry strips following a drying process. After the dry strips are formed, the active oxygen in the strips are monitored at initial, 5 days, 9 days and 16 days under room temperature.

The formula of the MPS slurry is listed in Table 7. The packaging materials used are polyethylene terephthalate (PET), polystyrene (PS) and polyethylene (PE).

TABLE 7
Formula of MPS Slurry:

| | Amount by |
|---|---|
| Component | wt. % |
| Caroat (45% MPS) | 2.2 |
| Water | q.s. (e.g., about 75%) |
| PVP | 10-20% |
| PEG 600 | 3-8% |
| Chelating agent | 0.1-1% |
| pH adjusting agent | 0.5-3% |
| Alkali metal pyrophosphate | 0.1-1% |

The retention rate of active oxygen in the dry strips was calculated and used to evaluate the MPS stability.

$$Retention Rate of Active Oxygen ( \% ) = \frac{Active Oxygen ( aged )}{Active Oxygen ( initial )} \times 100 \%$$

Although the active oxygen continuously decreases for all three packaging material options, the PET option demonstrates a larger drop compared to the PE and PS options. Without being bound by theory, this is believed to be because the unsaturated carbonyl groups in PET possibly react with MPS and consume more active oxygen in the strip. The reaction between PET and MPS is confirmed by the FTIR analysis (data not shown). Compared to the untreated PET, MPS-treated PET shows absorption decreases at 1710 cm⁻¹ and 723 cm⁻¹, and absorption increases at 800 cm⁻¹, which is believed to demonstrate the C═O structure in PET is broken by MPS.

### Example 5

A further study is conducted to evaluate the role of the humectants in the slurry composition. Five slurry formulas are prepared according to the following table and dried to form strips:

| Ingredient | Formula 5-1 | Formula 5-2 | Formula 5-3 | Formula 5-4 | Formula 5-5 |
|---|---|---|---|---|---|
| Caroat (45% MPS) | 2.2% | 2.2% | 2.2% | 2.2% | 2.2% |
| PEG-600 | 0% | 2% | 6.5% | 0% | 0% |
| Glycerin | 0% | 0% | 0% | 2% | 6.5% |
| PVP | 25% | 25% | 25% | 25% | 25% |
| NaOH, 10% Aq. | 1.6% | 1.6% | 1.6% | 1.6% | 1.6% |
| Water | Q.S. | Q.S. | Q.S. | Q.S. | Q.S. |

Each slurry formula contains either 2% or 6.5% of PEG-600 or glycerin as humectant, and the formulas are otherwise the same. Each formula is dried to form a strip. Strip stability is determined by measuring active oxygen content of the strip at initial formulation, after 5 days, and after 8 days, of storage at room temperature. The results are shown in the following table (expressed as percentage of initial active oxygen content):

| Formula | AO at day 5 (% of initial AO) | AO at day 8 (% of initial AO) |
|---|---|---|
| 5-1 | 97.96 | 96.44 |
| 5-2 | 95.56 | 91.31 |
| 5-3 | 91.65 | 84.65 |
| 5-4 | 96.37 | 89.19 |
| 5-5 | 86.60 | 76.51 |

The results show that strips prepared from a slurry formulas with 2% of each humectant have better stability than strips prepared from a slurry formula with the same humectant at 6.5%. In addition, at the same percentage of humectant in the slurry formula, strips with PEG-600 as humectant have better stability than strips with glycerin as humectant. While lower amounts of humectants are thus preferable, in other experiments it is found that a complete absence of humectant results in dried strips that are too brittle to use. The data suggest that the total amount of humectant is of primary importance, with amounts of less than 6% preferable, and amounts less than 4% or less than 2% more preferable, while the choice of humectant is of secondary importance, but PEG-type humectants (such as PEG-600) are preferable to polyhydric alcohol humectants (such as glycerin).

### Example 6

A further study is conducted to study the stability of MPS strips protected by backing layers made of different materials, including polyethylene terephthalate (PET), polystyrene (PS), and polyethylene (PE). An aqueous slurry according to Example 4 is prepared and coated onto a backing layer of PET, PS or PE, and then the product is dried. The slurry dries to become the hydratable adhesive film. Product stability is determined by measuring active oxygen content of the strip at initial formulation, after 5 days, after 9 days, and after 16 days, of storage at room temperature. It is unexpectedly found that the strips are substantially more stable when prepared on a PS or PE backing, compared to a PET backing. Without being bound by theory, it is believed that the functional groups in the PET polymer react with the potassium peroxymonosulfate during aging. Furthermore, it is believed that PS may be a difficult polymer to work with as the backing layer, because it does not form thin sheets easily as other polymers.

The invention has been described above with reference to illustrative Examples, but it is to be understood that the invention is not limited to the disclosed embodiments. Alterations and modifications that would occur to one of skill in the art upon reading the specification are also within the scope of the invention, which is defined in the appended claims.

1. An intermediate composition (e.g., an aqueous slurry) for the formulation of a tooth whitening strip comprising a hydratable adhesive film, wherein the intermediate composition comprises an inorganic salt of peroxymonosulfate.

2. The composition of claim 1, wherein the salt of peroxymonosulfate is potassium peroxymonosulfate.

3. The composition of claim 1, wherein the potassium peroxymonosulfate is provided as a triple salt of potassium peroxymonosulfate, potassium hydrogen sulfate and potassium sulfate, optionally wherein the triple salt comprises about 45%-50% by weight of potassium peroxymonosulfate, e.g., 47% or 49% by weight of potassium peroxymonosulfate.

4. The composition of claim 1, wherein the composition comprises an effective amount of peroxymonosulfate salt which is 0.01% to 5%, by weight of the composition.

5. The composition of claim 1, wherein the inorganic salt of peroxymonosulfate is the only oxidizing agent present in the composition.

6. The composition of claim 1, wherein the composition comprises one or more water-soluble or water-swellable polymers, including anionic polymers and/or neutral polymers.

7. The composition of claim 1, wherein the composition comprises one or more humectants selected from: polyethylene glycols (such as PEG-200, PEG-300, PEG-400, PEG-500, PEG-600, PEG-800, PEG-1000, PEG-1600, PEG-2000), polypropylene glycols, polyoxyethylene-polyoxypropylene copolymers (PEG-PPG, including block copolymers, triblock copolymers (poloxamers, such as poloxamer 407), and random copolymers, such as PEG/PPG-116/66 and PEG/PPG-38/8), ethanol, glycerin, propylene glycol, sorbitol, and xylitol.

8. The composition of claim 7, wherein the composition comprises one or more humectants selected from polyethylene glycols, PEG-PPG copolymers, and glycerin.

9. The composition of claim 7, wherein the composition comprises at least one polyethylene glycol, e.g., selected from PEG-200, PEG-300, PEG-400, PEG-500, PEG-600, PEG-800, PEG-1000, PEG-1600, and PEG-2000.

10. The composition of claim 9, wherein the composition comprises PEG-600.

11. The composition of claim 1, wherein the composition further comprises one or more polymer selected from polysaccharides (e.g., cellulose derivatives, for example carboxymethyl cellulose, hydroxyethyl cellulose, hydroxypropyl cellulose, hydroxypropyl methyl cellulose, ethyl cellulose, microcrystalline cellulose; or polysaccharide gums, for example xanthan gum, guar gum, or carrageenan gum, pectins, karaya gum); chitosans; dextrans; polyvinyl pyrrolidone (PVP), such as cross-linked PVP; hyaluronic acid and sodium hyaluronates; synthetic anionic polymeric polycarboxylates, such as copolymers of maleic anhydride or acid with another polymerizable ethylenically unsaturated monomer, preferably methyl vinyl ether (e.g., copolymers in a 1:4 to 4:1 ratio of maleic anhydride/acid to methyl vinyl ether); polyphosphonic acids and polyphosphonates; cross-linked carboxyvinyl copolymers; polyacrylic acid or polyacrylate polymers; polyacrylamides, such as (2-hydroxypropyl)methacrylamide; polyamines; polyvinyl alcohols; polyvinyl pyrrolidine-polyvinyl acetate copolymers (PVP-VA); polyoxazolines, such as poly(2-alkyl-2-oxazolines), isopropyl substituted polyoxazolines; and quaternary ammonium polymers.

12. The composition of claim 1, wherein the composition comprises one or more polyvinylpyrrolidone (PVP) polymers, e.g., linear and/or cross-linked PVP polymers.

13. The composition of claim 1, wherein the composition consists of water, PVP, potassium peroxymonosulfate triple salt (e.g., Caroat®), and one or more of polyethylene glycol (e.g., PEG-600), PEG-PPG copolymer (e.g., Poloxamer 105), and glycerin.

14. The composition of claim 1, wherein the composition is an aqueous slurry that comprises or consists of:

| | Amount by |
|---|---|
| Component | wt. % |
| MPS (e.g., provided as | 0.5-2% (e.g., 1%) |
| Caroat ®, 45% MPS) | |
| Water | q.s. (e.g., about 75%) |
| PVP | 10-20% |
| PEG 600 | 3-8% |
| Chelating agent | 0.1-1% |
| pH adjusting agent | 0.5-3% |
| Alkali metal pyrophosphate | 0.1-1% |

or
wherein the composition is an aqueous slurry that comprises or consists of:

| | Amount by |
|---|---|
| Component | wt. % |
| MPS (e.g., provided as | 0.3-1.5% |
| Caroat ®, 45% MPS) | (e.g., 0.6%) |
| Water | q.s. (e.g., about 60-80%) |
| PVP (e.g., PVP K90 and/or | 15-30% |
| PVP K29/32) | (e.g., 25%) |
| PEG (e.g., PEG 600) | 0.5-5% (e.g., 2%) |
| pH adjusting agent (e.g. NaOH) | 0.5-3% |

or

| | Amount by |
|---|---|
| Component | wt. % |
| MPS (e.g., provided as | 0.5-3% (e.g., 0.9%) |
| Caroat ®, 45% MPS) | |
| Water | q.s. (e.g., about 60-80%) |
| PVP (e.g., PVP K90 and/or | 15-30% |
| PVP K29/32) | (e.g., 25%) |
| PEG (e.g., PEG 600) | 0.5-5% (e.g., 2%) |
| pH adjusting agent (e.g. NaOH) | 0.5-3% |

15. The composition of claim 1, wherein the composition is an aqueous slurry.

16. A tooth whitening strip comprising a hydratable adhesive film, wherein the strip comprises or is prepared from an intermediate composition according to claim 1.

17. A tooth whitening strip according to claim 16, wherein the hydratable adhesive film of the strip comprises or consists of:

| | Amount by |
|---|---|
| Component | wt. % |
| MPS (e.g., provided as | 0.5-3% (e.g., 0.9-1.1%) |
| Caroat ®, 45% MPS) | |
| Water | q.s. (e.g., about 0-15%) |
| PVP (e.g., PVP K90 and/or | 50- 90% |
| PVP K29/32) | (e.g., 75-85%) |
| PEG (e.g., PEG 600) | 2-10% (e.g., 4-8%) |
| pH adjusting agent (e.g. NaOH) | 1-10% (e.g., 5%) |

18. The tooth whitening strip according to claim 17, wherein the strip further comprises an inert paper or plastic backing layer to which the hydratable adhesive film is adhered or affixed, and/or an inert removable paper or plastic protective film adhered to the hydratable adhesive film, optionally, wherein the protective film and/or the backing strip are comprised of wax paper, coated paper, treated paper, polyethylene, polystyrene, polypropylene, polyvinyl chloride, polyvinyl acetate, polymethyl methacrylate, polytetrafluoroethylene, polyesters (e.g., polyethylene terephthalate), polyimides, polycarbonates, or any combination thereof.

19. A method for producing a tooth whitening strip comprising the steps of (a) applying the composition of claim 1, to a backing layer, and (b) drying the composition to the backing layer to form the hydratable adhesive layer of the strip (e.g., by removing water and other volatile ingredients).

20. (canceled)

21. A method of whitening teeth, wherein the method comprises applying a strip according to claim 17 to one or more teeth.
